(19) United States
(12) Patent Application Publication
(10) Pub. No.: US 2024/0076261 A1
(43) Pub. Date: Mar. 7, 2024

(54) METHOD FOR PREPARING HIGH-BULK-DENSITY IBUPROFEN SPHERICAL CRYSTAL AND PRODUCT THEREOF

(71) Applicant: ZHEJIANG NHU COMPANY LTD., SHAOXING, Zhejiang Province (CN)

(72) Inventors: JINLONG LIU, SHAOXING, ZHEJIANG PROVINCE (CN); SHUMIN LV, SHAOXING, ZHEJIANG PROVINCE (CN); RUIJUN HU, SHAOXING, ZHEJIANG PROVINCE (CN); XUEZHONG HE, SHAOXING, ZHEJIANG PROVINCE (CN); MENGLIANG DU, SHAOXING, ZHEJIANG PROVINCE (CN); XIAOCHUN WANG, SHAOXING, ZHEJIANG PROVINCE (CN); ZIYU WANG, SHAOXING, ZHEJIANG PROVINCE (CN); LANYING LIANG, SHAOXING, ZHEJIANG PROVINCE (CN)

(21) Appl. No.: 18/270,725

(22) PCT Filed: Mar. 24, 2022
(86) PCT No.: PCT/CN2022/082699

§ 371 Date: Jun. 30, 2023

(30) Foreign Application Priority Data
Apr. 23, 2021 (CN) 202110440101.0

(51) Int. Cl. C07C 51/43

(52) U.S. Cl. CPC C07C 51/43; C07B 2200/13

(57) ABSTRACT

The present invention discloses a method for preparing an ibuprofen spherical crystal having high bulk density and a product thereof. The method includes: (1) heating ibuprofen until a molten liquid state is reached; and (2) pressurizing the molten liquid state of ibuprofen, dropping the molten liquid state of ibuprofen into a crystallizer filled with water through a liquid distributor for crystallization under stirring conditions until the molten liquid state of ibuprofen is completely dropped, lowering the rotation speed of stirring to allow crystal growing, and finally, subjecting a resulting crystal slurry to post-treatment to obtain the ibuprofen spherical crystal having high bulk density. The ibuprofen spherical crystal has a bulk density of 0.50-0.70 g/mL, a tap density of 0.63-0.89 g/mL, a median particle size of 300-1,000 μm and an angle of repose of 22-29°. The method of the present invention features that the process thereof is simple, and that no additives are used, such that the method is suitable for industrial production. The ibuprofen spherical crystal prepared has regular crystal habit, smooth crystal surface, good fluidity, high bulk density and high product purity. The quality of a product meets requirements of CP2020, EP9.8, USP41 and other pharmacopoeias.

### TECHNICAL FIELD

The present invention relates to the technical field of synthesis of medicines, and in particular to a method for preparing an ibuprofen spherical crystal having high bulk density and a product thereof.

### BACKGROUND TECHNOLOGY

Ibuprofen (CAS: 15687-27-1) has a chemical name of 2-methyl-4-(2-methylpropyl)phenylacetic acid, and is also known as Banufeng or isobutylphenylpropionic acid. The ibuprofen is a nonsteroidal anti-inflammatory drug widely used in clinical application with a history of more than 50 years. The ibuprofen has a molecular formula of C₁₃H₁₈O₂ and a molecular weight of 206.29, and is usually a white crystalline powder having a melting point of 75-77° C. The ibuprofen is insoluble in water and easily soluble in ethanol, chloroform, ether, acetone and other solvents. The ibuprofen is mainly used for relieving sprain and contusion, strain, headache, low back pain, postoperative pain and the like, and has the effects of relieving pain and reducing inflammation. The drug has been recorded in pharmacopoeias of America, Britain, the European Union, Japan and other countries since 1980s. As various preparations of the ibuprofen are almost in the OTC medication scope in various countries in the world, the ibuprofen has become one of best-selling over-the-counter drugs in the world and has been listed as three pillar products of antipyretic and analgesic drugs together with aspirin and paracetamol.

Most of ibuprofen bulk drug powders sold on the market have poor fluidity, poor tableting ability and strong adhesion to a mold during processing of solid preparations. Due to these defects, drug tablets or tableted substances have low strength, and a high content of an auxiliary material is usually required to be added or a complex wet granulation process is used, so that the production cost is increased. The fluidity of the ibuprofen powder can be effectively improved by increasing the stacking density of the ibuprofen and the sphericity of crystals. The ibuprofen produced by existing processes basically has needle-shaped crystal habit and a bulk density of 0.3-0.65 g/mL, and the angle of repose of the powder is 33-40°.

According to a Chinese patent CN102311335A, a process for preparing an ibuprofen bulk drug having high bulk density is disclosed. The process includes dropping an ethanol solution of ibuprofen into a cooled crystallization tank for cooling and crystallization; after the dropping is completed, adding water for further crystallization; and finally, conducting standing for crystallization, followed by centrifugation and drying to obtain a product having a bulk density of 0.55-0.65 g/mL and needle-shaped crystal habit. Since a high-temperature saturated solution is rapidly cooled, the supersaturation is too high, and crystals are relatively small.

According to an American patent U.S. Pat. No. 4,476,248A, an improved method for preparing crystalline ibuprofen is provided. The method includes preparing an ibuprofen/alcohol solvent into a saturated solution first; conducting cooling to a crystallization point; and then, adding a crystal seed, and conducting cooling continuously for crystallization to obtain a product having a bulk density of 0.4-0.62 g/mL, an average particle size of equal to or greater than 18 μm, long rod-shaped crystal habit and a crystal aspect ratio of equal to or less than 4:1.

According to a patent CN109956860A, a method for preparing an ibuprofen spherical crystal is introduced. The method includes preparing an ibuprofen/water mixed solution at 75-85° C. first; then conducting cooling for crystallization; adding a surfactant with a mass fraction of 0.02-0.53%, and conducting stirring continuously for 0.5-5 h to agglomerate a crystal into a ball; and then, conducting filtration, washing and drying to obtain the ibuprofen spherical crystal. The product has an average particle size of 500-1,000 μm, and crystal particles are round and have high fluidity, an angle of repose of 29-31° and a tap density of 0.47-0.55 g/mL. Since the pharmacopoeias in various countries have high requirements on the impurity content of ibuprofen bulk drugs, for example, the single impurity content of ibuprofen bulk drugs is required to be equal to or less than 0.05% according to a European pharmacopoeia EP10.0, excessive impurity residues of the surfactant are likely to be caused by the method.

According to an Indian patent IN229699B, a method for preparing a dense spherical ibuprofen agglomerate is disclosed. The method includes dissolving an ibuprofen particle in a first solvent (such as isopropanol) to obtain an ibuprofen solution first; adding a second solvent (such as water) for mixing with the ibuprofen solution; after the temperature is maintained at 10° C. for a certain period of time, heating a resulting mixture; then, adding a third solvent (such as isopropyl acetate) to obtain a slurry; and finally, conducting stirring to obtain the dense spherical ibuprofen agglomerate having a stacking density of 0.20-0.49 g/mL and an angle of repose of 23-29°. According to the method, a variety of solvents are used, the process is complex to control, the cost is high, and industrial production is not facilitated.

### SUMMARY OF THE INVENTION

In view of the above problems of the prior art, the present invention discloses a method for preparing an ibuprofen spherical crystal having high bulk density. The process is simple, and no additives are used, such that the method is suitable for industrial production. The ibuprofen spherical crystal prepared has regular crystal habit, smooth crystal surface, good fluidity, high bulk density and high product purity. The quality of a product meets requirements of CP2020, EP9.8, USP41 and other pharmacopoeias.

Specific technical solutions are as follows:

A method for preparing an ibuprofen spherical crystal having high bulk density includes:
(1) heating ibuprofen until a molten liquid state is reached; and
(2) pressurizing the molten liquid state of ibuprofen, dropping the molten liquid state of ibuprofen into a crystallizer filled with water through a liquid distributor for crystallization under stirring conditions until the molten liquid state of ibuprofen is completely dropped, lowering the rotation speed of stirring to allow crystal growing, and finally, subjecting a resulting crystal slurry to post-treatment to obtain the ibuprofen spherical crystal having high bulk density.

In the preparation process disclosed by the present invention, the ibuprofen (having a melting point of 75-77° C.) is heated to a temperature higher than the melting point to form a molten liquid first, and then tiny droplets with uniform distribution are formed through the liquid distributor and fully dispersed in the water by a high-shear stirrer. Tiny liquid distribution holes (0.2-1.0 mm) of the liquid distributor provide an environment for forming the tiny droplets. Through rotational dropping by the liquid distributor, uniform distribution of the droplets is ensured. As the water is cooled in advance, the tiny droplets dispersed in the water are rapidly cooled, crystallized and precipitated. By means of high-shear stirring, the adhesion and agglomeration of the tiny droplets and crystals can be effectively prevented, and smooth crystal surfaces are facilitated. Then, by means of crystal growing treatment, growth of crystals is further promoted, and finally, the ibuprofen spherical crystal having high bulk density is formed.

In step (1), preferably, the heating is conducted to a temperature of 77-85° C.

In step (2):
the molten liquid state of ibuprofen is pressurized by compressed air, nitrogen or inert gas, or by a fluid conveying device such as a pump, and the inert gas is selected from common types in the field.

By adjusting the pressurizing pressure, the dropping rate of the droplets into the crystallizer and the distribution of the droplets can be adjusted. Within a certain pressure range, when the pressure is higher, the dropping rate is higher, the distribution of the droplets is relatively more uniform, and the particle size of the crystal is relatively smaller. Preferably, the pressurizing pressure is 0.15-0.40 MPa. It has been found by a test that a prepared product has many adhesive crystals when the pressure is too low; and a product has irregular crystals when the pressure is too high. Further preferably, the pressurizing is conducted to 0.15-0.25 MPa.

The molten liquid state of ibuprofen is dropped into the crystallizer through the liquid distributor. The size and distribution of the ibuprofen droplets falling to the surface of the water is controlled by the liquid distributor. The size of the liquid distribution holes of the liquid distributor and the pressure of the ibuprofen droplets entering the distributor are adjusted, so as to achieve the purpose of adjusting the size and distribution of the ibuprofen droplets falling to the surface of the water. It has been found by a test that when the diameter of the liquid distribution holes of the liquid distributor is smaller, the pressure of the ibuprofen droplets entering the distributor is higher, and the particle size of the obtained crystal is smaller. Preferably, the diameter of the liquid distribution holes of the liquid distributor is 0.2-1.0 mm; further preferably, the diameter is 0.3-0.7 mm; and more preferably, the diameter is 0.5-0.7 mm.

The liquid distributor is selected from a rotary ball type distributor, a spiral nozzle type distributor, and a shower head type rotary distributor. Preferably, a rotary ball type distributor is used. By means of the rotary ball type distributor, uniform distribution of the droplets can be better realized, so that the particle size and distribution of the crystal are uniform.

Preferably, the mass ratio of the water to the ibuprofen is (2.0-7.0):1. It has been found by a test that when too little water is used, an adhesive crystal is likely to be caused, so that the crystal surface is not smooth; and when too much water is used, dispersion of the droplets falling into the water in a main water phase is affected, so that the particle size of the crystal is nonuniform. Further preferably, the mass ratio is (2.0-4.0):1.

The water may be selected from ordinary water, deionized water, and purified water, and is preferably purified water.

The temperature of the water in the dropping process is further required to be controlled. It has been found by a test that when the temperature is too high, the droplets falling into the water cannot precipitate a crystal quickly, and an adhesive crystal is likely to be caused, so that the particle size and distribution of the crystal are nonuniform. Preferably, the temperature of the water is controlled at 1-30° C.; and further preferably, the temperature of the water is controlled at 1-15° C.

The crystallizer is further internally provided with a stirrer, and a single-stage or multi-stage impeller stirrer may be used. Preferably, a multi-stage impeller stirrer is used.

In the process of dropping for crystallization, the stirrer needs to rotate at high speed, and by means of high shear force, uniform dispersion of the droplets in a semi-emulsified state is ensured. Preferably, the rotation speed is 250-800 rpm. It has been found by a test that when the rotation speed is too low, a product has few crystals with irregular appearance, and the product has a large angle of repose; and when the rotation speed is too high, a product has a large number of broken crystals and irregular crystal habit. Further preferably, the rotation speed is 300-600 rpm.

In the process of crystal growing, by lowering the rotation speed of stirring, breaking of crystals caused by violent collision and friction of the crystals can be avoided, so that further growth of the crystals is facilitated, and the crystals have more regular crystal habit and more uniform particle size. Preferably, the rotation speed is lowered to 10-100 rpm. It has been found by a test that when the rotation speed is too low during crystal growing, a product has many adhesive crystals; and when the rotation speed is too high, a product has many broken crystals and irregular crystal habit. Further preferably, the rotation speed is lowered to 10-80 rpm.

Further preferably, in step (2):
the mass ratio of the water to the ibuprofen is (2.0-3.0):1;
the molten liquid state of ibuprofen is pressurized to 0.15-0.20 MPa;
in the dropping process, the temperature of the water is controlled at 5-10° C.;
the diameter of the liquid distributing holes of the liquid distributor is 0.5-0.6 mm;
in the process of dropping for crystallization, the rotation speed of stirring is 300-350 rpm; and in the process of crystal growing, the rotation speed of stirring is lowered to 20-30 rpm.

At this time, the ibuprofen spherical crystal prepared has regular crystal habit, smooth crystal surface, high bulk density, smaller angle of repose and better fluidity.

The post-treatment includes centrifugal filtration, washing and drying.

The present invention further discloses an ibuprofen spherical crystal having high bulk density prepared by the method. The ibuprofen spherical crystal has a bulk density of 0.50-0.70 g/mL, a tap density of 0.63-0.89 g/mL, a median particle size of 300-1,000 μm and an angle of repose of 22-29°.

Compared with the prior art, the present invention has the following beneficial effects:

(1) The process is simple, and no additives are used, such that the method is suitable for industrial production. A product has high purity, and the quality of the product meets requirements of CP2020, EP9.8, USP41 and other pharmacopoeias.

(2) The ibuprofen spherical crystal prepared has regular crystal habit, smooth crystal surface, good fluidity, high bulk density and good tableting ability, and requirements of solid preparations for processing and molding are met.

### BRIEF DESCRIPTION OF DRAWINGS

FIG. 1 is a diffraction pattern of an ibuprofen spherical crystal powder prepared in Example 1 obtained by XRD.

FIG. 2 is a morphology pattern of ibuprofen spherical crystals prepared in Example 1 obtained by scanning electron microscopy.

### DETAILED DESCRIPTION OF EMBODIMENTS

The present invention is further described in detail below in combination with examples, but embodiments of the present invention are not limited thereto.

Detection Method

1. Method for Determining the Bulk Density:

(1) A clean and dry 100 mL measuring cylinder is taken and weighed to obtain a mass (m₀, g).

(2) A sample sifted through a 20-mesh sieve is slowly loaded into the measuring cylinder to (90±5) mL, the measuring cylinder and the sample are weighed to obtain a total mass (m₁, g), accurate to 0.1 g, the surface of a powder is carefully leveled, and the volume (V₁, mL) of the powder is read.

(3) Calculation is conducted as follows: bulk density=(m₁−m₀)/V₁.

2. Method for Determining the Tap Density:

The tap density is an increased stacking density obtained after a container containing a sample powder is mechanically beaten. In the present invention, the tap density is determined by a method I in 2.9.34 of a European pharmacopoeia EP9.8.

3. Method for Determining the Angle of Repose:

The angle of repose usually refers to the maximum angle formed between a free slope and a horizontal plane of a stacked layer of a powder. A smaller angle of repose indicates that the friction is smaller and the fluidity is better, and it is generally believed that the fluidity is good when θ is equal to or less than 30°. The fluidity of a powder has great influence on the weight difference and normal operation of granules, capsules, tablets and other preparations. In the present invention, the angle of repose of a crystal is determined by a test method according to the standard GB 11986-89.

### Example 1

(1) First, 5 kg of ibuprofen was put into a melting tank and heated to 80° C. for melting.

(2) 10 kg of purified water was added into a crystallizer, a jacket of the crystallizer was opened to realize cooling with freezing brine, and the purified water was cooled to 5° C.

(3) A double-layer paddle type stirrer in the crystallizer was turned on, where the rotation speed was controlled at 300 rpm.

(4) The melting tank in step (1) was pressurized to 0.15 MPa with nitrogen, and a resulting ibuprofen molten liquid was dropped from the melting tank into the crystallizer through a rotary ball type liquid distributor, where the diameter of liquid distribution holes of the rotary ball type liquid distributor was 0.6 mm. Droplets were rotatably sprayed and then uniformly dropped to the surface of water, and a flow control valve was adjusted to make the whole batch of liquid completely dropped within 1.5 hours.

(5) In the dropping process, the temperature in the crystallizer was controlled at 5° C. to ensure crystallization while the ibuprofen molten liquid was dropped until the whole batch of ibuprofen molten liquid was completely added. The rotation speed was lowered to 20 rpm to realize crystal growing at a temperature of 5° C. for 0.5 hour. Then, a resulting crystal slurry was subjected to centrifugal filtration, washing and drying to obtain 4.9 kg of ibuprofen crystals.

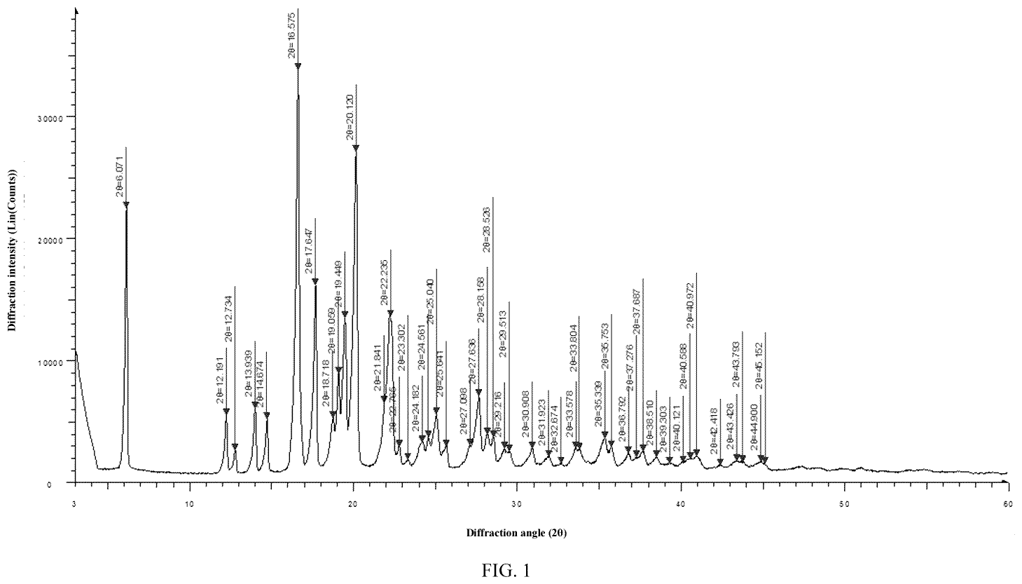

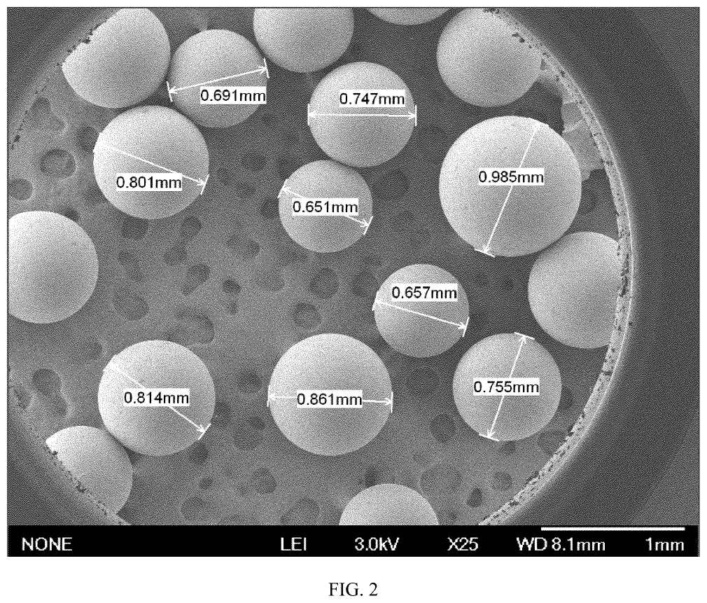

An XRD powder diffraction pattern and an SEM morphology pattern of a product prepared in this example are as shown in FIG. 1 and FIG. 2 respectively. It can be seen that the crystals are stable ibuprofen crystals having spherical and regular crystal habit and smooth crystal surfaces. According to tests, the product has a bulk density of 0.68 g/mL, a tap density of 0.86 g/mL, a median particle size of 770 μm, an angle of repose of 250 and good fluidity, and the quality meets requirements of CP2020, EP9.8, USP41 and other pharmacopoeias.

### Examples 2-9 and Comparative Examples 1-10

On the basis of the preparation process in Example 1, some process parameters are adjusted below in Examples 2-9 and Comparative Examples 1-10. Specific adjusted process parameters as well as the bulk density, angle of repose and crystal habit of finally prepared products are listed in Table 1 below.

TABLE 1

| | Adjustment | Bulk | Tap | Angle of | |
|---|---|---|---|---|---|
| | of process | density of | density of | repose of | |
| Number | parameters | products/g/mL | products/g/mL | products/° | Crystal habit of products |
| Example 2 | In the | 0.51 | 0.63 | 29 | The median particle size |
| | dropping for | | | | was 420 μm, spherical |
| | crystallization, | | | | crystals with smooth |
| | the rotation | | | | crystal surfaces were |
| | speed of | | | | obtained, and about 6% of |
| | stirring was | | | | broken crystals were |
| | 750 rpm. | | | | found. |
| Example 3 | In the crystal | 0.66 | 0.83 | 27 | The median particle size |
| | growing, the | | | | was 672 μm, spherical |
| | rotation speed | | | | crystals with smooth |
| | was 90 rpm. | | | | crystal surfaces were |
| | | | | | obtained, and about 5% of |
| | | | | | broken crystals were |
| | | | | | found. |
| Example 4 | The diameter | 0.52 | 0.66 | 28 | The median particle size |
| | of the liquid | | | | was 225 μm, most |
| | distributing | | | | spherical crystals with |
| | holes of the | | | | smooth crystal surfaces |
| | liquid | | | | were obtained, and about |
| | distributor | | | | 9% of broken crystals |
| | was 0.2 mm. | | | | were found. |
| Example 5 | The diameter | 0.70 | 0.89 | 29 | The median particle size |
| | of the liquid | | | | was 986 μm, basically |
| | distributing | | | | spherical crystals with |
| | holes of the | | | | smooth crystal surfaces |
| | liquid | | | | were obtained, and about |
| | distributor | | | | 7% of adhesive crystals |
| | was 1.0 mm. | | | | were found. |
| Example 6 | The diameter | 0.66 | 0.83 | 22 | The median particle size |
| | of the liquid | | | | was 716 μm, basically |
| | distributing | | | | spherical crystals with |
| | holes of the | | | | regular crystal habit and |
| | liquid | | | | smooth crystal surfaces |
| | distributor | | | | were obtained, and almost |
| | was 0.5 mm. | | | | no broken crystals and |
| | | | | | adhesive crystals were |
| | | | | | found. |
| Example 7 | The molten | 0.62 | 0.79 | 29 | The median particle size |
| | liquid was | | | | was 515 μm, basically |
| | pressurized at | | | | spherical crystals were |
| | a pressure of | | | | obtained, and about 3% of |
| | 0.40 MPa. | | | | adhesive crystals were |
| | | | | | found. |
| Example 8 | The mass ratio | 0.65 | 0.82 | 28 | The median particle size |
| | of the water to | | | | was 680 μm, spherical |
| | the ibuprofen | | | | crystals with smooth |
| | was 7.0:1. | | | | crystal surfaces were |
| | | | | | obtained, and about 4% of |
| | | | | | adhesive crystals were |
| | | | | | found. |
| Example 9 | The mass ratio | 0.65 | 0.83 | 23 | The median particle size |
| | of the water to | | | | was 703 μm, spherical |
| | the ibuprofen | | | | crystals with smooth |
| | was 3.0:1. | | | | crystal surfaces were |
| | | | | | obtained, and almost no |
| | | | | | broken crystals and |
| | | | | | adhesive crystals were |
| | | | | | found. |
| Comparative | The | 0.69 | 0.79 | 40 | Crystals with rough |
| Example 1 | temperature | | | | crystal surfaces and |
| | of the water | | | | irregular crystal habit |
| | was 35° C. | | | | appearance were |
| | | | | | obtained, and about 19% |
| | | | | | of adhesive crystals were |
| | | | | | found. |
| Comparative | In the | 0.70 | 0.84 | 38 | The median particle size |
| Example 2 | dropping for | | | | was 1,200 μm, few |
| | crystallization, | | | | crystals with irregular |
| | the rotation | | | | appearance were |
| | speed of | | | | obtained, and about 6% of |
| | stirring was | | | | adhesive crystals were |
| | 200 rpm. | | | | found. |
| Comparative | In the | 0.43 | 0.55 | 39 | The median particle size |
| Example 3 | dropping for | | | | was 176 μm, crystals with |
| | crystallization, | | | | irregular crystal habit |
| | the rotation | | | | were obtained, and about |
| | speed of | | | | 21% of broken crystals |
| | stirring was | | | | were found. |
| | 1,000 rpm. | | | | |
| Comparative | In the crystal | 0.72 | 0.82 | 42 | The median particle size |
| Example 4 | growing, the | | | | was 1,183 μm, and about |
| | rotation speed | | | | 14% of adhesive crystals |
| | was 5 rpm | | | | were found. |
| Comparative | In the crystal | 0.63 | 0.80 | 37 | The median particle size |
| Example 5 | growing, the | | | | was 614 μm, crystals with |
| | rotation speed | | | | irregular crystal habit |
| | was 200 rpm | | | | were obtained, and about |
| | | | | | 24% of broken crystals |
| | | | | | were found. |
| Comparative | The diameter | 0.72 | 0.82 | 41 | The median particle size |
| Example 6 | of the liquid | | | | was 1,164 μm, and about |
| | distributing | | | | 18% of adhesive crystals |
| | holes of the | | | | were found. |
| | liquid | | | | |
| | distributor | | | | |
| | was 1.5 mm. | | | | |
| Comparative | The molten | 0.71 | 0.84 | 39 | The median particle size |
| Example 7 | liquid was | | | | was 1,231 μm, and about |
| | pressurized at | | | | 26% of adhesive crystals |
| | a pressure of | | | | were found. |
| | 0.05 MPa. | | | | |
| Comparative | The molten | 0.53 | 0.67 | 38 | The median particle size |
| Example 8 | liquid was | | | | was 403 μm, and a part of |
| | pressurized at | | | | spherical crystals and a |
| | a pressure of | | | | part of fine and irregular |
| | 0.50 MPa. | | | | crystals accounting for |
| | | | | | about 25% were obtained. |
| Comparative | The mass ratio | 0.73 | 0.83 | 42 | The median particle size |
| Example 9 | of the water to | | | | was 1,326 μm, crystals |
| | the ibuprofen | | | | with relatively rough |
| | was 1.0:1. | | | | crystal surfaces were |
| | | | | | obtained, and about 31% |
| | | | | | of adhesive crystals were |
| | | | | | found. |
| Comparative | The mass ratio | 0.58 | 0.73 | 39 | The median particle size |
| Example 10 | of the water to | | | | was 410 μm, crystals with |
| | the ibuprofen | | | | relatively rough crystal |
| | was 9.0:1. | | | | surfaces were obtained, |
| | | | | | and about 28% of broken |
| | | | | | crystals and irregular |
| | | | | | crystals were found. |

After observing the data in the above Table 1, it can be seen that when ibuprofen spherical crystals having a bulk density of 0.50-0.70 g/mL, a tap density of 0.63-0.89 g/mL, a median particle size of 300-1,000 μm and an angle of repose of 22-29°, a variety of parameters in the method are required to be adjusted simultaneously to fall within appropriate value ranges respectively.

### Example 10

A tableting experiment is carried out by using the ibuprofen spherical crystals having high bulk density prepared by the above crystallization process as a main component and adding pharmaceutical auxiliary materials according to a conventional prescription. Ibuprofen tablets can be prepared by a dry method or a wet method, and an optional general process is as follows.

10 kg of ibuprofen spherical crystals, 1.5 kg of microcrystalline cellulose, 0.31 kg of crosslinked sodium carboxymethyl cellulose, 0.10 kg of micropowder silica and 0.07 kg of magnesium stearate were weighed, put into a mixer for mixing, and then sifted through a 20-mesh standard sieve to obtain a premix having excellent fluidity. The premix was continuously tableted by a rotary tablet press at a pressure of 6 kN for 10 hours until no sticking phenomenon was found. Obtained tablets are smooth in appearance, indicating that the spherical crystals having high bulk density prepared according to the present invention have excellent tableting performance.

### Examples 11-13 and Comparative Examples 11-13

On the basis of Example 10, the type of crystals was adjusted below in Examples 11-13 and Comparative Examples 11-13. Specific adjustments are listed in Table 2 below.

TABLE 2

| Number | Type of crystals | Tableting performance |
|---|---|---|
| Example 11 | Spherical crystals, having an angle of | After continuous pressing |
| | repose of 22°, obtained in Example 6 | for 10 hours, no sticking |
| | were used in the prescription. | phenomenon was found. |
| Example 12 | Spherical crystals, having an angle of | After continuous pressing |
| | repose of 27°, obtained in Example 3 | for 10 hours, no sticking |
| | were used in the prescription. | phenomenon was found. |
| Example 13 | Spherical crystals, having an angle of | After continuous pressing |
| | repose of 29°, obtained in Example 7 | for 10 hours, a sticking |
| | were used in the prescription. | phenomenon was found. |
| Comparative | Commercially available needle-shaped | After continuous pressing |
| Example 11 | ibuprofen crystals, having an angle of | for 3 hours, a sticking |
| | repose of 32°, were used in the | phenomenon was found. |
| | prescription. | |
| Comparative | Commercially available needle-shaped | After continuous pressing |
| Example 12 | ibuprofen crystals, having an angle of | for 1.5 hours, a sticking |
| | repose of 39°, were used in the | phenomenon was found. |
| | prescription. | |
| Comparative | Crystals, having an angle of repose of | After continuous pressing |
| Example 13 | 40°, obtained in Comparative Example 1 | for 2.0 hours, a sticking |
| | were used in the prescription. | phenomenon was found. |

After observing the data in Table 2, it can be seen that the spherical crystals having high bulk density prepared according to the present invention have excellent tableting performance and are conducive to production of preparations.

1. A method for preparing an ibuprofen spherical crystal having high bulk density, comprising:
(1) heating ibuprofen until a molten liquid state is reached; and
(2) pressurizing the molten liquid state of ibuprofen, dropping the molten liquid state of ibuprofen into a crystallizer filled with water through a liquid distributor for crystallization under stirring conditions until the molten liquid state of ibuprofen is completely dropped, lowering the rotation speed of stirring to allow crystal growing, and finally, subjecting a resulting crystal slurry to post-treatment to obtain the ibuprofen spherical crystal having high bulk density.

2. The method for preparing an ibuprofen spherical crystal having high bulk density according to claim 1, wherein in step (1), the heating is conducted to a temperature of 77-85° C.

3. The method for preparing an ibuprofen spherical crystal having high bulk density according to claim 1, wherein in step (2), the molten liquid state of ibuprofen is pressurized to 0.15-0.40 MPa.

4. The method for preparing an ibuprofen spherical crystal having high bulk density according to claim 1, wherein in step (2), the mass ratio of the water to the ibuprofen is (2.0-7.0):1;
and in the dropping process, the temperature of the water is controlled at 1-30° C.

5. The method for preparing an ibuprofen spherical crystal having high bulk density according to claim 1, wherein in step (2), the diameter of liquid distributing holes of the liquid distributor is 0.2-1.0 mm.

6. The method for preparing an ibuprofen spherical crystal having high bulk density according to claim 1, wherein in step (2), in the process of dropping for crystallization, the rotation speed of stirring is 250-800 rpm; and in the process of crystal growing, the rotation speed of stirring is lowered to 10-100 rpm.

7. The method for preparing an ibuprofen spherical crystal having high bulk density according to claim 1, wherein in step (2),
the mass ratio of the water to the ibuprofen is (2.0-4.0):1;
the molten liquid state of ibuprofen is pressurized to 0.15-0.25 MPa;
in the dropping process, the temperature of the water is controlled at 1-15° C.;
the diameter of the liquid distributing holes of the liquid distributor is 0.3-0.7 mm;
in the process of dropping for crystallization, the rotation speed of stirring is 300-600 rpm;
and in the process of crystal growing, the rotation speed of stirring is lowered to 10-80 rpm.

8. The method for preparing an ibuprofen spherical crystal having high bulk density according to claim 1, wherein in step (2), the post-treatment comprises centrifugal filtration, washing and drying.

9. An ibuprofen spherical crystal having high bulk density prepared by the method according to claim 1, wherein the ibuprofen spherical crystal has a bulk density of 0.50-0.70 g/mL, a median particle size of 300-1,000 μm and an angle of repose of 22-29°.

10. The ibuprofen spherical crystal having high bulk density prepared by the method according to claim 9, wherein the ibuprofen spherical crystal has a tap density of 0.63-0.89 g/mL.
